(19) United States
(12) Patent Application Publication
(10) Pub. No.: US 2013/0172879 A1
(43) Pub. Date: Jul. 4, 2013

(54) RENAL NERVE MODULATION MEDICAL DEVICES

(71) Applicant: BOSTON SCIENTIFIC SCIMED, INC., Maple Grove, MN (US)

(72) Inventors: DEREK C. SUTERMEISTER, EDEN PRAIRIE, MN (US); TIM A. OSTROOT, COKATO, MN (US); JOEL R. MUNSINGER, BLAINE, MN (US); JOSEF V. KOBLISH, SUNNYVALE, CA (US); ELIAS A. KHOURY, CHAMPLIN, MN (US); JAMES M. ANDERSON, FRIDLEY, MN (US)

(73) Assignee: BOSTON SCIENTIFIC SCIMED, INC., MAPLE GROVE, MN (US)

(21) Appl. No.: 13/715,839

(22) Filed: Dec. 14, 2012

Related U.S. Application Data
(60) Provisional application No. 61/581,468, filed on Dec. 29, 2011.

(51) Int. Cl. A61B 18/14

(52) U.S. Cl. CPC A61B 18/1492
USPC 606/41

(57) ABSTRACT

Medical devices and methods for making and using medical devices are disclosed. An example medical device may include a renal nerve modulation device. The renal nerve modulation device may include an elongate shaft having proximal region and a distal region. An ablation member may be coupled to the distal region. The distal region may have a distal inner diameter. The proximal region may have a proximal inner diameter that is smaller than the distal inner diameter. A ribbon may be disposed within the distal region of the shaft. The ribbon may have a proximal end and a distal end. The proximal end of the ribbon may extend into the proximal region of the shaft. The distal end of the ribbon may be disposed adjacent to or otherwise coupled to the ablation member.

### CROSS-REFERENCE TO RELATED APPLICATIONS

This application claims priority under 35 U.S.C. §119 to U.S. Provisional Application Ser. No. 61/581,468, filed Dec. 29, 2011, the entirety of which is incorporated herein by reference.

### TECHNICAL FIELD

The present disclosure pertains to medical devices, and methods for manufacturing medical devices. More particularly, the present disclosure pertains to renal nerve modulation medical devices and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device may include a renal nerve modulation device. The renal nerve modulation device may include an elongate shaft having proximal region and a distal region. An ablation member may be coupled to the distal region. The distal region may have a distal inner diameter. The proximal region may have a proximal inner diameter that is smaller than the distal inner diameter. A ribbon may be disposed within the distal region of the shaft. The ribbon may have a proximal end and a distal end. The proximal end of the ribbon may extend into the proximal region of the shaft. The distal end of the ribbon may be coupled to the ablation member.

Another example renal nerve modulation device may include an elongate shaft having a distal region and an inner surface. An ablation member may be coupled to the distal region. The inner surface may be stepped radially outward so that the distal region has a larger inner diameter than portions of the shaft proximal of the distal region. A ribbon may be disposed within the distal region of the shaft. The ribbon may have a proximal end and a distal end. An ablation member lead may be coupled to the ablation member and may extend proximally along the inner surface of the shaft to a proximal end of the shaft.

An example method for renal nerve modulation may include providing a renal nerve modulation device. The renal nerve modulation device may include an elongate shaft having a distal region and an inner surface, an ablation member coupled to the distal region, a ribbon disposed within the distal region of the shaft, the ribbon having a proximal end and a distal end, and an ablation member lead coupled to the ablation member extending proximally along the inner surface of the shaft to a proximal end of the shaft. The inner surface may be stepped radially outward so that the distal region has a larger inner diameter than portions of the shaft proximal of the distal region. The method may also include advancing the renal nerve modulation device through a blood vessel to a position adjacent to a renal nerve and actuating the ablation member to ablate the renal nerve.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view illustrating an example renal nerve modulation system;

FIG. 2 is a schematic view illustrating the location of the renal nerves relative to the renal artery;

FIG. 3 is a partial cross-sectional side view of an example medical device;

FIG. 4 is a partial cross-sectional side view of another example medical device;

FIG. 5 is a side view of a portion of an example shaft for use with a medical device;

FIG. 6 is a side view of a portion of another example shaft for use with a medical device;

FIG. 7 is a side view of a portion of another example shaft for use with a medical device;

FIG. 8 is a side view of a portion of another example shaft for use with a medical device; and

FIG. 9 is a side view of a portion of another example shaft for use with a medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless clearly stated to the contrary.

Certain treatments may require the temporary or permanent interruption or modification of select nerve function. One example treatment is renal nerve ablation which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

Because the nerves are hard to visualize, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

While the devices and methods described herein are discussed relative to renal nerve modulation through a blood vessel wall, it is contemplated that the devices and methods may be used in other applications where nerve modulation and/or ablation are desired. The term modulation refers to ablation and other techniques that may alter the function of affected nerves.

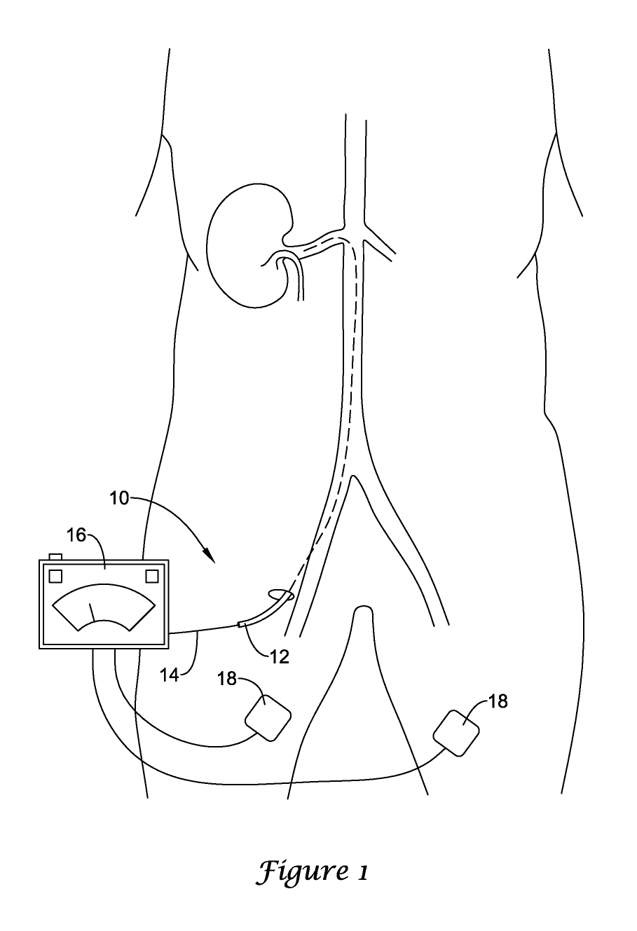

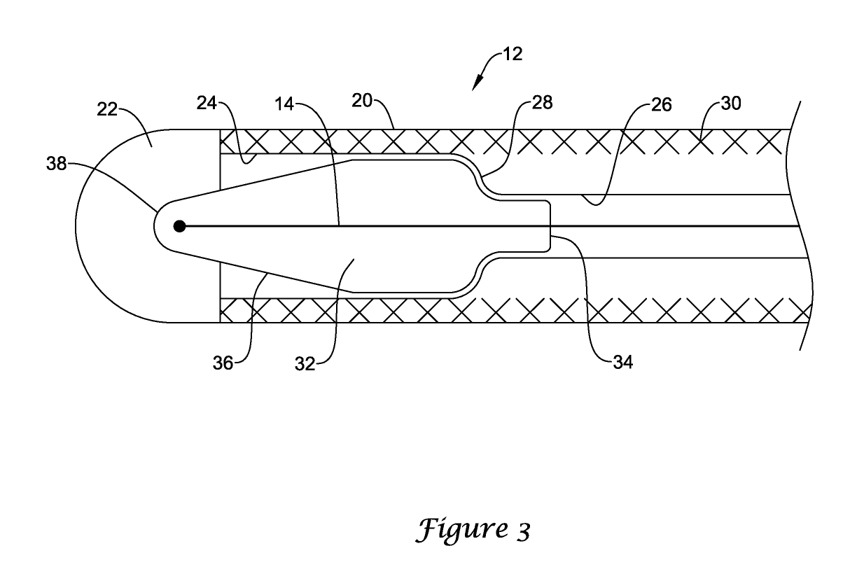

FIG. 1 is a schematic view of an example renal nerve modulation system 10 in situ. System 10 may include a renal ablation medical device 12 and one or more conductive element(s) 14 for providing power to medical device 12. A proximal end of conductive element(s) 14 may be connected to a control and power element 16, which supplies necessary electrical energy to activate one or more electrodes (e.g., ablation member 22 as shown in FIG. 3) disposed at or near a distal end of medical device 12. When suitably activated, the electrodes are capable of ablating adjacent tissue. The terms electrode and electrodes may be considered to be equivalent to elements capable of ablating adjacent tissue in the disclosure which follows. In some instances, return electrode patches 18 may be supplied on the legs or at another conventional location on the patient's body to complete the circuit.

Control and power element 16 may include monitoring elements to monitor parameters such as power, temperature, voltage, amperage, impedance, pulse size and/or shape and other suitable parameters, with sensors mounted along catheter, as well as suitable controls for performing the desired procedure. In some embodiments, power element 16 may control a radio frequency (RF) electrode. The electrode may be configured to operate at a frequency of approximately 460 kHz. It is contemplated that any desired frequency in the RF range may be used, for example, from 450-500 kHz. It is further contemplated that additionally and/or other ablation devices may be used as desired, for example, but not limited to resistance heating, ultrasound, microwave, and laser devices and these devices may require that power be supplied by the power element 16 in a different form.

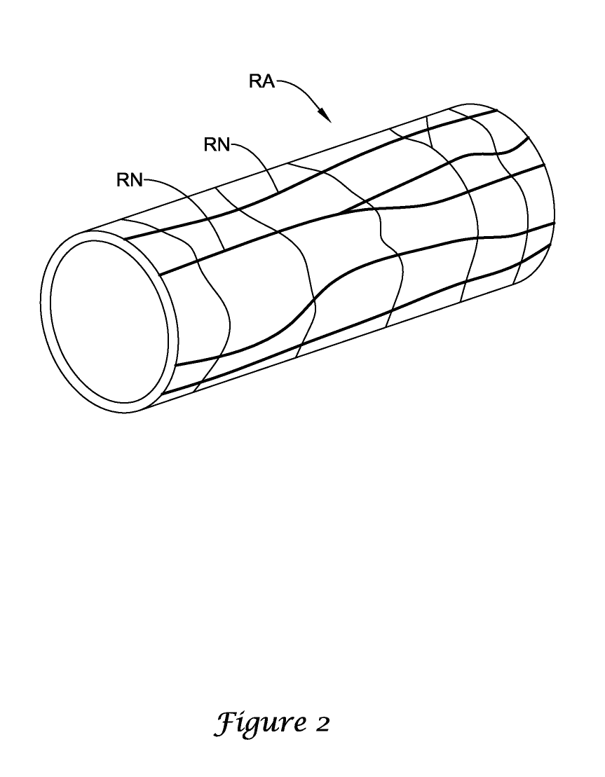

FIG. 2 illustrates a portion of the renal anatomy in greater detail. More specifically, the renal anatomy includes renal nerves RN extending longitudinally along the lengthwise dimension of renal artery RA and generally within or near the adventitia of the artery. The human renal artery wall is typically about 1 mm thick of which 0.5 mm is the adventitial layer. As will be seen in the figure, the circumferential location of the nerves at any particular axial location may not be readily predicted. Nerves RA are difficult to visualize in situ and so treatment methods may desirably rely upon ablating multiple sites to ensure nerve modulation.

FIG. 3 is a partially cut away side view of medical device 12. Here, some of the structural features of medical device 12 can be seen. For example, medical device 12 may include an elongate shaft 20. An ablation member or electrode 22 may be attached to shaft 20. Ablation member 22 may be formed at or otherwise form a distal tip of shaft 20. Other locations are contemplated. In general, ablation member 22 may be configured to ablate target tissue at or near a body lumen. For example, ablation member 22 may be used to ablate renal nerves adjacent to a renal artery. This may include advancing medical device through the vasculature to the renal artery and actuating ablation member 22 to ablate renal nerves positioned adjacent to the renal arty.

Ablation member 22 may include suitable ablation structure(s) such as one or more RF electrodes, PT/IR electrodes, other electrodes and/or transducers (thermal, ultrasonic, laser, microwave, etc.) or the like. In embodiments where a plurality of electrodes or ablation members are utilized, the ablation member may be arranged in any suitable way including being spaced longitudinally along shaft 20, being circumferentially oriented about shaft 20, or in any other suitable arrangement. Ablation member 22 may vary and may include a number of structures such as a plurality of wires (e.g., two wires) that connect with electrode wire 14 and, ultimately, control and power element 16. Electrode wire 14 may be soldered to a side slot on the ablation member 22, for example. Ablation member 22 may also include other structures and/or features associated typically associated with ablation (e.g., thermal ablation) such as a temperature monitoring member (not shown), which may take the form of a thermocouple or thermistor. In at least some embodiments, a thermistor including two thermistor wires may be disposed adjacent to ablation member 22.

Shaft 20 may take the form of a metallic and/or polymer tube. In at least some embodiments, shaft 20 may form or define an outer surface of medical device 12. Shaft 20 may also have an inner surface and/or inner diameter that varies along the length of shaft 20. For example, shaft 20 may include a distal inner diameter region 24 and a proximal inner diameter region 26. Distal inner diameter region 24 may have a larger inner diameter than proximal inner diameter region 26. A stepped region 28 may be defined between distal inner diameter region 24 and proximal inner diameter region 26. Stepped region 28, as the name suggested, may form a step or transition in inner diameter. Alternatively, stepped region 28 may be tapered or otherwise form a gradual transition between distal inner diameter region 24 and proximal inner diameter region 26 (and may also help to gradually transition the flexibility of shaft 20 between distal inner diameter region 24 and proximal inner diameter region 26). Shaft 20 may also include a reinforcing member 30. Reinforcing member 30 may provide structural reinforcement to shaft 20 and may help transmit torque along the length of shaft 20. In some embodiments, reinforcing member 30 includes a braid. In some of these and in alternative embodiments, reinforcing member 30 may include a coil or other reinforcing structure. Reinforcing member 30 may extend along only a portion of the length of shaft 20 or reinforcing member 30 may extend along substantially the full length of shaft 20 (e.g., to the distal end of shaft 20). In some embodiments, at least a portion of reinforcing member 30 may be encapsulated or embedded within shaft 20. In some of these and in alternative embodiments, at least a portion of reinforcing member 30 may be disposed along an inner and/or outer surface of shaft 20.

At least a portion of reinforcing member 30 may also extend through substantially the entire radial thickness of shaft 20. In some of these and in alternative embodiments, at least a portion of reinforcing member 30 may extend only part-way through the radial thickness of shaft 20. For example, along distal inner diameter region 24, reinforcing member 30 may extend through a majority or substantially all of the radial thickness of shaft 20 whereas along proximal inner diameter region 26, reinforcing member 30 may extend through only a portion of the radial thickness of shaft 20. These are just examples and alternative arrangements are contemplated including arrangements where reinforcing member 30 extends through a part of or substantially all of the radial thickness of distal inner diameter region 24, proximal inner diameter region 26, or both.

Shaft 20 may also include a pull wire or steering mechanism (not shown) that can be used to deflect or otherwise “steer” shaft 20 and/or ablation member 22. This may allow the shape or configuration of shaft 20 to be altered. This may include steering or directing medical device 12 so that ablation member 22 is situated as desired within the renal artery. This may include laying a distal portion of shaft 20 (including ablation member 22) flat against the vessel wall of the renal artery. The precise form and configuration of the pull wire or steering mechanism may vary and include essentially any suitable mechanism.

Some ablation medical devices may include an interior coil that is used for compression resistance of the shaft or ablation member. However, the presence of the coil may necessitate the shaft having a larger interior or inner diameter in order to accommodate the coil. Medical device 12 may include one or more structures that help provide shaft 20 with improved compression resistance. For example, stepped region 28 may be sized and/or configured to abut or otherwise engage a ribbon member 32 (and/or other portions of medical device) coupled to shaft 20. This may provide resistance to compressive forces that may be applied to ablation member 22 and/or the distal portion of shaft 20. In addition, because stepped region 28 is generally formed as a deflection in shaft 20, a compression resistance coil is not necessary (e.g., at least some of the medical devices 12 are free of a coil such as an interior compression resistance coil).

The absence of a compression resistance coil may also allow the overall profile of medical device 12 to be reduced. For example, medical device 12 may have an outer diameter and/or outer profile is less than about 6 Fr (e.g., less than about 0.079 inches), or an outer diameter and/or outer profile that is about 3-5 Fr (e.g. about 0.039 to 0.066 inches), or an outer diameter and/or outer profile that is about 4 Fr (e.g., about 0.053 inches). These are just examples. Having a lower profile may be desirable for a number of reasons. For example, having a lower profile may help reduce trauma that may be associated with navigating medical device 12 through the vasculature, may allow medical device 12 to be used with smaller patients including children, may allow medical device 12 to reach smaller portions of the vasculature (including the neurovasculature), etc.

The absence of a compression resistance coil may also simplify manufacturing of medical device 12. For example, securing the coil in place and/or other manufacturing processes may be avoided. In addition, manufacturing costs may be reduced by eliminating the coil (and may be further reduced if the coil would have included a coating such as polytetrafluoroethylene, which may be relatively expensive).

As indicated above, ribbon member 32 may also be coupled to shaft 20. In at least some embodiments, ribbon member 32 is disposed within shaft 20. This may include at least a portion of ribbon member 32 being disposed within distal inner diameter region 24. Ribbon member 32 may include a proximal end or proximal end region 34. Proximal end 34 may be disposed within proximal inner diameter region 26. In some embodiments, proximal end 34 is attached to proximal inner diameter region 26. For example, proximal end 34 may be mechanically attached to, thermally bonded with, glued, welded, brazed, or otherwise attached to proximal inner diameter region 26. This may include attaching proximal end 34 to the inner surface of shaft 20 along proximal inner diameter region 26. In other embodiments, proximal end 34 may not be attached to proximal inner diameter region 26.

Ribbon member 32 may have a flattened or ribbon-like shape where the width is greater than the thickness. For example, ribbon member 32 may have a width (e.g., a maximum width) in the range of about 0.01 to 0.05 inches, or about 0.02 to 0.04 inches, or about 0.025 to 0.035 inches. The thickness of ribbon member 32 may be in the range of about 0.001 to 0.008 inches, or about 0.002 to 0.006 inches, or about 0.003 to 0.005 inches. The compression resistance included within medical device (e.g., at stepped region 28) may also allow the length of ribbon member 32 to be reduced. This may allow help to reduce the curvature length and/or radius so that ablation member 22 can be brought into the desired contact, for example, with the renal artery. For example, the reduced radius of curvature may allow ablation member 22 to be positioned flat against the wall of the renal artery during ablation. In at least some embodiments, the length of ribbon member 32 may be about 0.25 to 1.75 inches, or about 0.30 to 1.6 inches, or about 0.33 to 1.5 inches.

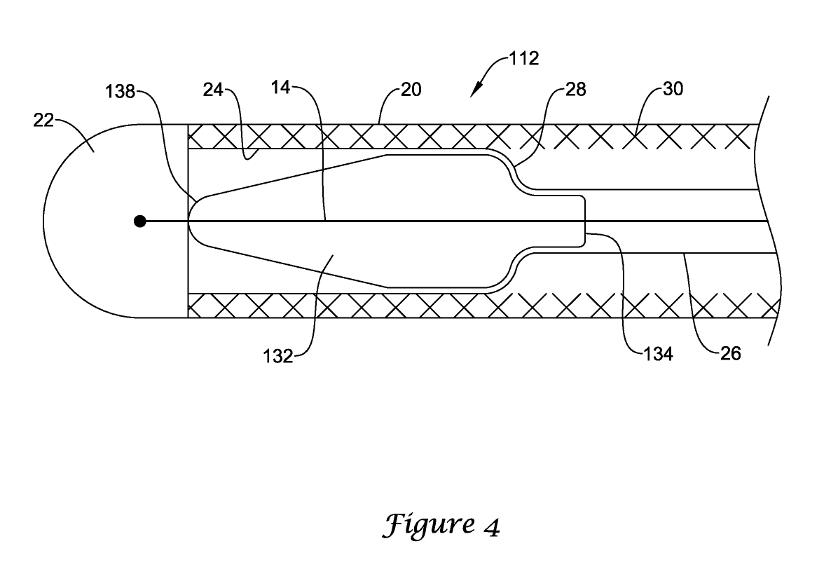

Ribbon member 32 may also include a tapered distal portion 36 that terminates in a distal end or distal end region 38. In at least some embodiments, distal end 38 may be attached to or otherwise bonded with ablation member 22. However, this is not intended to be limiting. Other embodiments are contemplated including embodiments where distal portion 36 of ribbon member 32 is not attached to ablation member 22 and/or where distal portion 36 is longitudinally spaced from ablation member 22. For example, FIG. 4 illustrates medical device 112 that includes a ribbon member 132 where the distal end 138 may abut or otherwise be disposed adjacent to ablation member 22. In these embodiments, distal end 138 may or may not be attached to ablation member 22. Proximal end 134 of ribbon member 132 may be secured to proximal inner diameter region 26 in a manner similar to what is described above for proximal end 34.

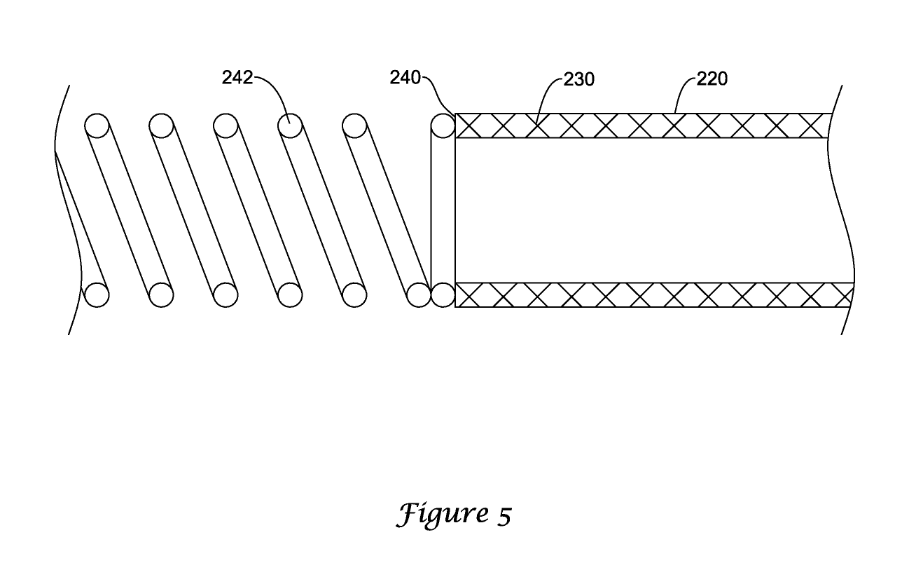

FIGS. 5-9 illustrate some additional variations contemplated for shaft 20 and any of these shaft variations may be utilized for any of the medical devices disclosed herein. For example, FIG. 5 illustrates shaft 220, which may be similar in form and function to other shafts disclosed herein. Shaft 220 may include reinforcing member 230. A distal coil 242 may be attached to or otherwise coupled with a distal end 240 of shaft 220. Coil 242 may extend distally from distal end 240 of shaft 220. In some embodiments, coil 242 may be positioned about ribbon member 32/132. In other embodiments, coil 242 may be disposed proximally and/or distally of ribbon member 32/132. Coil 242 may have an open pitch as shown or coil 242 may have a closed or partially closed pitch.

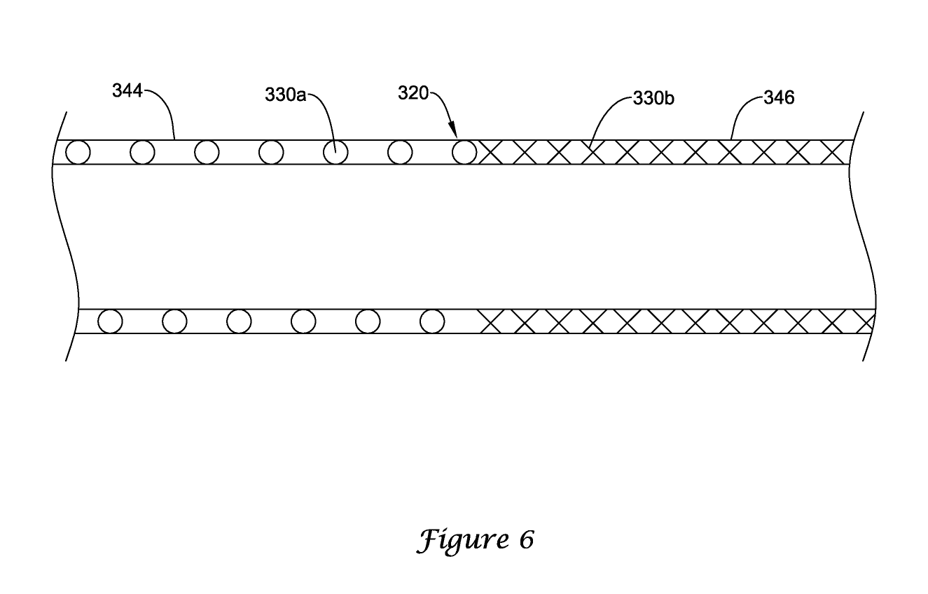

FIG. 6 illustrates shaft 320, which may be similar in form and function to other shafts disclosed herein. Shaft 320 may include a first portion 344 having a first reinforcing member 330a coupled therewith and a second portion 346 having a second reinforcing member 330b coupled therewith. First portion 344 may be a distal portion or a proximal portion (relative to second portion 346). In at least some embodiments, reinforcing members 330a/330b may be different structures. For example, reinforcing member 330a may take the form of a coil and reinforcing member 330b may take the form of a braid. In other embodiments, reinforcing members 330a/330b may be the same structure (e.g., both braids, coils, etc.) and may be formed from a single monolith of material. In some of these and in other embodiments, reinforcing members 330a/330b may be a differently sized or arranged version of the same structure (e.g., braids with a different pic counts, coils with a different pitches, etc.).

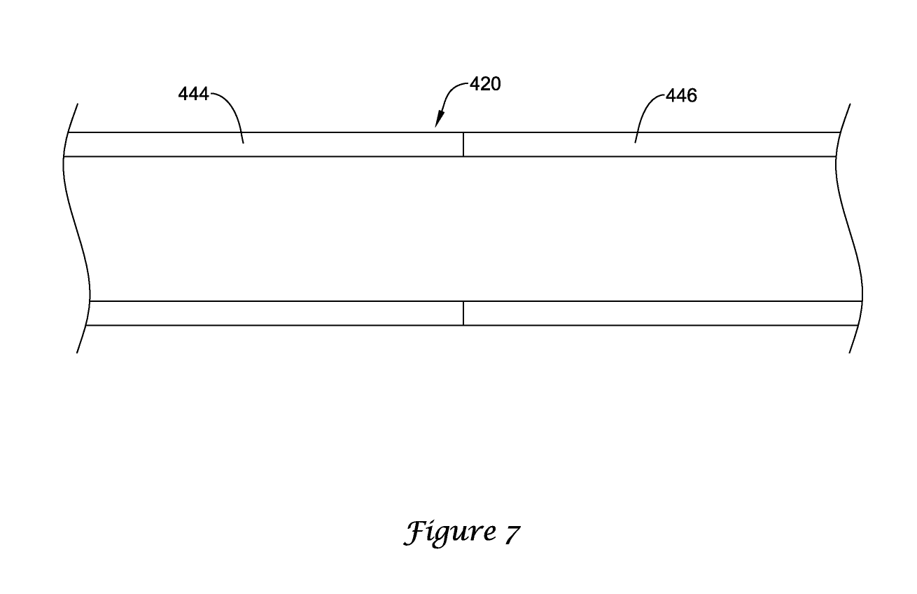

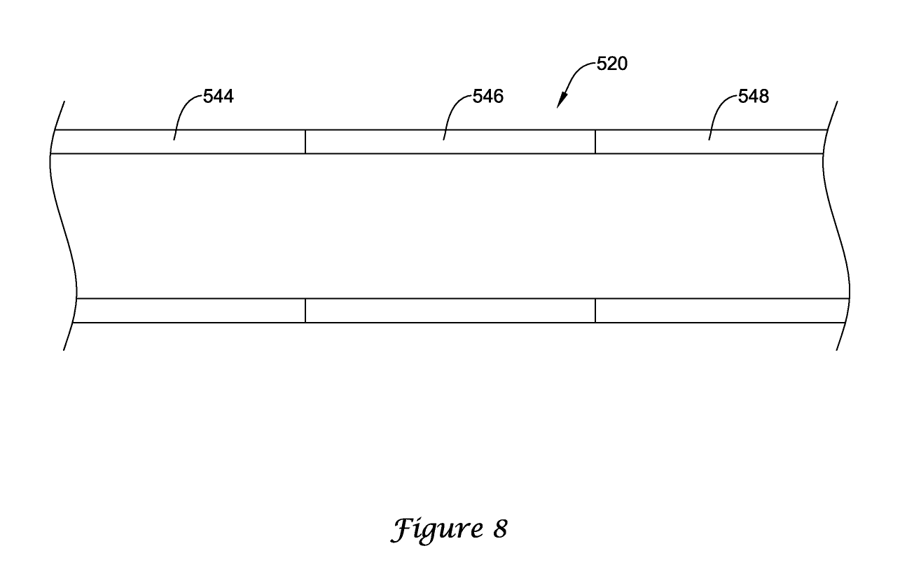

FIG. 7 illustrates shaft 420, which may be similar in form and function to other shafts disclosed herein. Shaft 420 may include first portion 444 and second portion 446. First portion 444 may be a distal portion or a proximal portion (relative to second portion 446). In at least some embodiments, portions 444/446 are formed from different polymers. For example, portion 444 may be formed from a first polymer having a first flexibility and portion 446 may be formed from a second polymer having a second flexibility different from the first flexibility. Shaft 420 is not intended to be limited to having just two portions 444/446. For example, FIG. 8 illustrates shaft 520, which may include first portion 544, second portion 546, and third portion 548. In at least some embodiments, two or more of portions 544/546/548 are formed from different polymers. For example, portion 544 may be formed from a first polymer having a first flexibility, portion 546 may be formed from a second polymer having a second flexibility different from the first flexibility, and portion 548 may be formed from a third polymer having a third flexibility different from the first flexibility (and/or different from the second flexibility). Other shafts are also contemplated that include more than three portions.

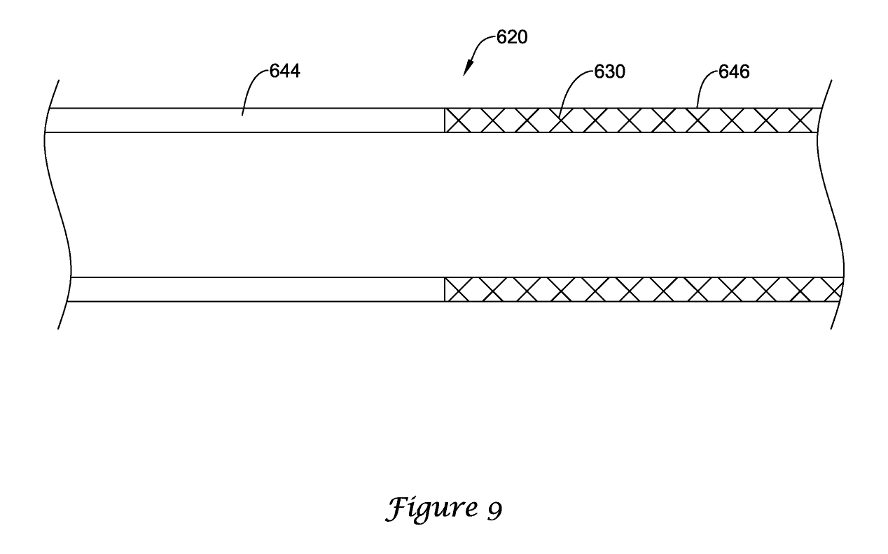

FIG. 9 illustrates shaft 620, which may be similar in form and function to other shafts disclosed herein. Shaft 620 may include first portion 644 and second portion 646. Reinforcing member 630 (e.g., a braid, coil, etc.) may be disposed along second portion 646. In some of these and in other embodiments, reinforcing member 630 may be disposed along essentially any portion or discrete section of shaft 620. First portion 644, however, may lack reinforcing member 630.

The materials that can be used for the various components of medical device 12 (and/or other medical devices disclosed herein) and the various shaft and/or members disclosed herein may include those commonly associated with medical devices. For simplicity purposes, the following discussion makes reference to shaft 20 and other components of medical device 12. However, this is not intended to limit the devices and methods described herein, as the discussion may be applied to other similar tubular members and/or components of tubular members or devices disclosed herein.

Shaft 20 and/or other components of medical device 12 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL° 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of shaft 20 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of medical device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of medical device 12 to achieve the same result.

In some embodiments, a degree of Magnetic Resonance Imaging (MRI) compatibility is imparted into medical device 12. For example, shaft 20 or portions thereof, may be made of a material that does not substantially distort the image and create substantial artifacts (i.e., gaps in the image). Certain ferromagnetic materials, for example, may not be suitable because they may create artifacts in an MRI image. Shaft 20, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of shaft 20 that may define a generally smooth outer surface for medical device 12. In other embodiments, however, such a sheath or covering may be absent from a portion of all of medical device 12, such that shaft 20 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical device 12 (including, for example, the exterior surface of shaft 20) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of shaft 20 or other portions of medical device 12. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A renal nerve modulation device, comprising:
an elongate shaft having proximal region and a distal region;
an ablation member coupled to the distal region;
wherein the distal region has a distal inner diameter;
wherein the proximal region has a proximal inner diameter that is smaller than the distal inner diameter;
a ribbon disposed within the distal region of the shaft, the ribbon having a proximal end and a distal end;
wherein the proximal end of the ribbon extends into the proximal region of the shaft; and
wherein the distal end of the ribbon is coupled to the ablation member.

2. The renal nerve modulation device of claim 1, wherein the shaft includes a reinforcing member.

3. The renal nerve modulation device of claim 2, wherein the reinforcing member includes a coil.

4. The renal nerve modulation device of claim 2, wherein the reinforcing member includes a braid.

5. The renal nerve modulation device of claim 2, wherein the reinforcing member extends to a distal end of the shaft.

6. The renal nerve modulation device of claim 2, wherein the reinforcing member extends to a position proximal of a distal end of the shaft.

7. The renal nerve modulation device of claim 1, wherein a coil is coupled to the distal region of the shaft and extends distally therefrom.

8. The renal nerve modulation device of claim 1, wherein the shaft includes two or more discrete polymer sections.

9. The renal nerve modulation device of claim 1, wherein an inner surface of the shaft is stepped radially inward along the proximal region of the shaft to define the proximal inner diameter.

10. The renal nerve modulation device of claim 1, wherein the proximal end of the ribbon is mechanically attached to the proximal region.

11. The renal nerve modulation device of claim 1, wherein the shaft includes an inner surface and wherein the inner surface of the shaft is free of a coil.

12. The renal nerve modulation device of claim 1, wherein the shaft has an outer diameter of 0.039 to 0.066 inches.

13. The renal nerve modulation device of claim 1, wherein the shaft has an outer diameter of 0.053 inches.

14. A renal nerve modulation device, comprising:
an elongate shaft having a distal region and an inner surface;
an ablation member coupled to the distal region;
wherein the inner surface is stepped radially outward so that the distal region has a larger inner diameter than portions of the shaft proximal of the distal region;
a ribbon disposed within the distal region of the shaft, the ribbon having a proximal end and a distal end; and
an ablation member lead coupled to the ablation member and extending proximally along the inner surface of the shaft to a proximal end of the shaft.

15. The renal nerve modulation device of claim 14, wherein the shaft includes a reinforcing member.

16. The renal nerve modulation device of claim 15, wherein the reinforcing member includes a braid, a coil, or both.

17. The renal nerve modulation device of claim 14, wherein a proximal end of the ribbon is mechanically attached to the inner surface of the shaft.

18. The renal nerve modulation device of claim 14, wherein the distal end of the ribbon is attached to the ablation member.

19. The renal nerve modulation device of claim 14, wherein the distal end of the ribbon is longitudinally spaced from the ablation member.

20. A method for renal nerve modulation, the method comprising:
providing a renal nerve modulation device, the renal nerve modulation device comprising:
an elongate shaft having a distal region and an inner surface,
an ablation member coupled to the distal region,
wherein the inner surface is stepped radially outward so that the distal region has a larger inner diameter than portions of the shaft proximal of the distal region,
a ribbon disposed within the distal region of the shaft, the ribbon having a proximal end and a distal end, and
an ablation member lead coupled to the ablation member and extending proximally along the inner surface of the shaft to a proximal end of the shaft;
advancing the renal nerve modulation device through a blood vessel to a position adjacent to a renal nerve; and
actuating the ablation member to ablate the renal nerve.
